(19) United States
(12) Patent Application Publication
(10) Pub. No.: US 2018/0317775 A1
(43) Pub. Date: Nov. 8, 2018

(54) AUXILIARY TOOL FOR SELECTIVELY VISUALIZING ARTERY

(71) Applicant: PLUSMED CORPORATION, Kochi-shi (JP)

(72) Inventors: Tetsuo SUMIDA, Kochi-shi (JP); Hiromi SATO, Kochi-shi (JP)

(73) Assignee: PLUSMED CORPORATION, Kochi-shi (JP)

(21) Appl. No.: 15/824,512

(22) Filed: Nov. 28, 2017

(30) Foreign Application Priority Data
May 5, 2017 (JP) 2017-091272

(51) Int. Cl. A61B 5/00; A61M 5/42

(52) U.S. Cl. CPC A61B 5/0086; A61B 5/0075; A61B 5/489; A61B 90/13; A61B 5/742; A61B 5/6824; A61M 5/427

(57) ABSTRACT

In order to puncture the artery running in parallel to the deep vein, a method or a tool for selectively visualizing the artery in a near-infrared image so as to form a near-infrared image of a portion to be punctured is provided. In the method for visualizing the artery by forming a near-infrared image of a portion of the artery to be punctured, the surface of the skin to be punctured is pressurized with a near-infrared light transparent material so as to collapse the deep vein running in parallel to the artery as well as the subcutaneous vein. An auxiliary tool for selectively visualizing the artery preferred for this pressurization is formed from a near-infrared light transparent material and has a notch through which a puncture needle can pass.

### TECHNICAL FIELD

The present invention relates to an auxiliary tool for selectively visualizing an artery, which is useful for selectively visualizing an artery when forming a near-infrared image of a portion of the artery to be punctured.

### BACKGROUND

In the inspection or medical treatment using a catheter, for example, in the cardiac catheter inspection, the artery is punctured, the guide wire is inserted, and the catheter is inserted. The portion to be punctured is, for example, the radial artery, the brachial artery, or the femoral artery. Of these arteries, the radial artery is suitable as a portion to be punctured because the radial artery can readily ensure hemostatic rest after the inspection and requires no constraint to the behavior of the patient.

In general, the running of the radial artery is estimated by palpation in order to puncture the radial artery. However, the puncture of the radial artery requires a high-level technique. If the puncture is difficult to perform, the puncture may be performed using an ultrasonography device. But, it is very intricate to manipulate the probe of the ultrasonography device while scanning the same, and moreover, the ultrasonography device requires relatively high costs.

On the other hand, the near-infrared light has a high permeability to the human body tissue such as the skin, fat, and muscle, but the hemoglobin in the blood absorbs the near-infrared radiation. With the help of this property, commercially available is a device which irradiates the surface of the skin with the near-infrared light to form a near-infrared image from the reflected light thereof, and displays the image on an eye-glass type terminal, thereby allowing the blood-vessel image to be observed as if the blood vessel is viewed through the surface of the skin (Eyes-On Glaass, Evena Medical Inc.). Another commercially available device projects, onto the skin surface, a near-infrared image formed by the reflected light of the near-infrared light with which the skin surface was irradiated, thereby visualizing the blood vessels under the skin (VeinViewer, Christie Medical Holdings Inc.). However, by the blood-vessel images formed by these devices, only the subcutaneous vein that is found within 2 to 3 mm under the skin can be visualized. Thus, these devices can be used to puncture the subcutaneous vein, but cannot be used to puncture the artery that is found at 5 to 10 mm under the skin. From such a clinical context, such a technique for visualizing various types of arteries, particularly, the radial artery in a simplified manner at relatively low costs has been awaited.

As a technique responding to this situation, such an artery visualization device has been suggested which irradiates the skin on the backside of the wrist with a near-infrared beam of light, and then receives the near-infrared beam transmitted through the wrist on the palm side of the wrist so as to form a near-infrared image of the artery of the wrist (Patent Literature 1 and Patent Literature 2). In this device, to prevent the image of the artery from becoming unclear due to the near-infrared beam being absorbed by the capillary network, the skin on the backside of the wrist is pressurized to collapse the capillary network under the skin of the backside of the wrist.

### CITATION LIST

### Patent Literature

Patent Literature 1: Japanese Patent No. 5626943

Patent Literature 2: Japanese Patent No. 6047847

### SUMMARY OF INVENTION

### Technical Problem

However, as disclosed in Patent Literatures 1 and 2, even if the skin on the backside of the wrist is pressurized to collapse the capillary network on the backside of the wrist, it may be difficult to accurately determine the position of an artery because a near-infrared image may show the artery and the deep vein running in parallel to the artery as diameter-increased blood-vessel since the artery image and the vein image are overlapped, or because a blood-vessel image of the subcutaneous vein on the palm side of the wrist may be overlapped with the blood-vessel image of the artery.

In this context, the present invention addresses the problem of selectively visualizing an artery in a near-infrared image at the time of forming a near-infrared image of a portion to be punctured when the artery is to be punctured, in particular, when the artery like the radial artery running in parallel to the deep vein is punctured.

### Solution to Problem

The inventor has found that in forming a near-infrared image of a portion of the artery to be punctured, when the artery, the deep vein running in parallel to the artery, and the subcutaneous vein on the puncture side are pressurized from the skin surface to be punctured, these veins will be collapsed, so that the artery can be selectively visualized so as to clearly identify the position of the artery. As a result, the inventor has conceived the present invention.

That is, the present invention provides an auxiliary tool for selectively visualizing an artery to be used when forming a near-infrared image of a portion of the artery to be punctured, the tool being formed from a near-infrared light transparent material and having a puncture part through which a puncture needle can pass.

Furthermore, the present invention provides a method for visualizing an artery by forming a near-infrared image of a portion of the artery to be punctured, the method including pressurizing, with a near-infrared light transparent material, a surface of the skin to be punctured so as to collapse the deep vein running in parallel to the artery, and the subcutaneous vein on the puncture side, thereby selectively visualizing the artery.

### Advantageous Effects of Invention

According to the method for visualizing the artery of the present invention, to visualize the artery by forming a near-infrared image of a portion of the artery to be punctured, the surface of the skin to be punctured is pressurized with a near-infrared light transparent material so as to collapse the deep vein running in parallel to the artery and the subcutaneous vein on the puncture side. At the time of the pressurization, the collapsed portion of the veins is not visually recognized in the near-infrared image. This allows the artery to be selectively visualized even if the artery and the veins cannot be distinguished from each other because the artery and the deep vein running in parallel to the artery are overlapped with each other causing a blood-vessel image of an increased diameter to be observed in a near-infrared image before the pressurization or because the blood-vessel image of the artery and the blood-vessel image of the subcutaneous vein on the puncture side are overlapped with each other. Furthermore, the pressurization of the skin surface causes the capillary network of the skin to be collapsed, thereby making the near-infrared image clearer. Thus, according to the method for visualizing an artery of the present invention, it is possible to readily find out the position to be punctured.

Furthermore, according to the auxiliary tool for selectively visualizing an artery of the present invention, it is possible to readily perform the method for visualizing an artery of the present invention.

### BRIEF DESCRIPTION OF DRAWINGS

FIG. 1A is a perspective view illustrating an auxiliary tool LA for selectively visualizing an artery according to an embodiment, and

FIG. 1B is a cross-sectional view illustrating the auxiliary tool 1A for selectively visualizing an artery according to the embodiment.

FIG. 2 is a perspective view illustrating an auxiliary tool 1B for selectively visualizing an artery according to an embodiment.

FIG. 3 is a perspective view illustrating an auxiliary tool 1C for selectively visualizing an artery according to an embodiment.

FIG. 4 is a perspective view illustrating an auxiliary tool 1D for selectively visualizing an artery according to an embodiment.

FIG. 5 is an explanatory view illustrating an auxiliary tool 1D in use for selectively visualizing an artery according to an embodiment.

FIG. 6A is a schematic cross-sectional view illustrating a portion of the radial artery to be punctured, before being pressurized, and FIG. 6E is a schematic cross-sectional view illustrating a portion of the radial artery to be punctured, at the time of being pressurized.

FIG. 7A is a view showing a near-infrared image before a portion of the radial artery to be punctured is pressurized, and FIG. 7B is a view showing a near-infrared image when a portion of the radial artery to be punctured is pressurized.

### DESCRIPTION OF EMBODIMENTS

The present invention will now be described in more detail with reference to the accompanying drawings. Note that throughout the drawings, the same reference signs denote the same or identical components.

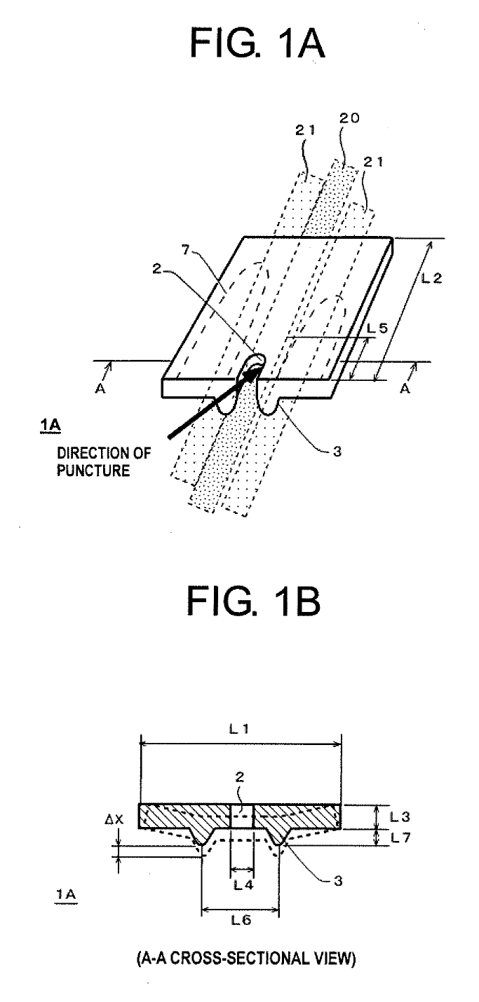

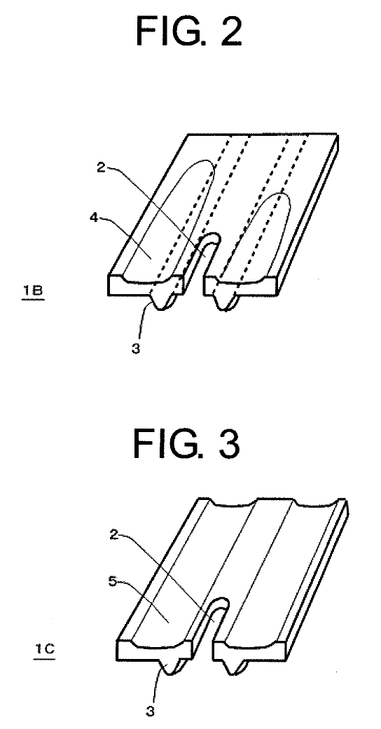

FIG. 1A is a perspective view illustrating an auxiliary tool 1A for selectively visualizing an artery according to an embodiment of the present invention, and FIG. 1B is a cross-sectional view thereof.

In irradiating a portion of the artery to be punctured with near-infrared light so as to form a near-infrared image of the portion to be punctured by a reflected beam or transmitted beam of the light, the auxiliary tool 1A, which is configured to selectively visualize the artery, is useful for selectively visualizing the artery in the near-infrared image. Here, the formation itself of the near-infrared image of the portion to be punctured can be performed by a well-known method, for example, the near-infrared image of a portion of the radial artery to be punctured may be formed by the methods disclosed in Patent Literatures 1 or 2.

This auxiliary tool 1A is formed from a near-infrared light transparent material. The wavelength of near-infrared light lies within a wavelength range which is absorbed by the hemoglobin in the blood and transmitted through the human body tissue such as the skin, fat, and muscle. More specifically, what is meant by the transparency of the near-infrared light is that the transmittance of near-infrared light at 840 nm to 950 nm is preferably 10% or greater, and more preferably 50% or greater. Furthermore, the auxiliary tool LA may also preferably be transparent in a visible light region so that with the auxiliary tool 1A placed on the skin of the portion to be punctured, the outer shape of the skin can be observed therethrough. Examples of the materials having such an optical property may include an acrylic resin, a methacrylic resin, and a polycarbonate resin.

The auxiliary tool of the present invention is not limited to a particular outer shape. However, the auxiliary tool 1A of this embodiment is formed from a generally rectangular plate-shaped material. The size of the auxiliary tool 1A can be determined so that the auxiliary tool 1A is depressed by a finger to thereby pressurize the skin to be punctured, and the deep vein 21 running in parallel to the artery 20 and the subcutaneous vein in the vicinity of the artery 20 can be readily collapsed. Specifically, the auxiliary tool 1A has a width L1 of preferably 20 mm to 80 mm, and more preferably 30 mm to 50 mm; a length L2 of preferably 30 mm to 150 mm, more preferably 40 mm to 80 mm; and a thickness L3 of preferably 0.5 mm to 4 mm, and more preferably 1 mm to 3 mm. As will be discussed later, as a method of using the auxiliary tool 1A, while the auxiliary tool 1A is being pushed with a finger of an operator to puncture the skin against the surface of the skin to be punctured, the position of the auxiliary tool 1A is displaced with the finger to preferably find the position at which the artery is selectively best visualized. However, a plate-shaped material excessively increased in size may make it difficult to displace the auxiliary tool 1A with the finger in this manner, and the pressing force exerted by the finger may be distributed, thereby making it difficult to strongly pressurize the vein in the vicinity of the portion to be punctured.

Furthermore, the plate-shaped material forming the auxiliary tool 1A may preferably be flexible so that even in the presence of a bone in the vicinity of the artery to be punctured, the auxiliary tool 1A can depress the artery and the deep vein running in parallel to the artery through the skin surface of the portion to be punctured. The level of the flexibility is preferably set such that when both ends of the plate-shaped material in the width direction are supported and the center thereof is depressed with a load of 5 kg, the plate-shaped material is curved as denoted by the broken line in FIG. 1B and the amount of distortion ΔX in the thickness direction of the plate is preferably about 0.1 mm to 1 mm. This enables the plate-shaped material to be adequately curved when the plate-shaped material is depressed with a finger at the center of the upper surface of the auxiliary tool 1A.

The auxiliary tool 1A has a notch 2 on a side edge of the plate-shaped material as a puncture part through which a puncture needle can pass. For the auxiliary tool of the present invention, a hole having no opening may be formed on a side edge of the plate-shaped material as a puncture part. However, the notch 2 may preferably be formed on the side edge of the plate-shaped material because the auxiliary tool 1A is pressed against the skin so that a puncture needle punctures, and after that, without drawing out the puncture needle, the auxiliary tool 1A can be taken off the skin.

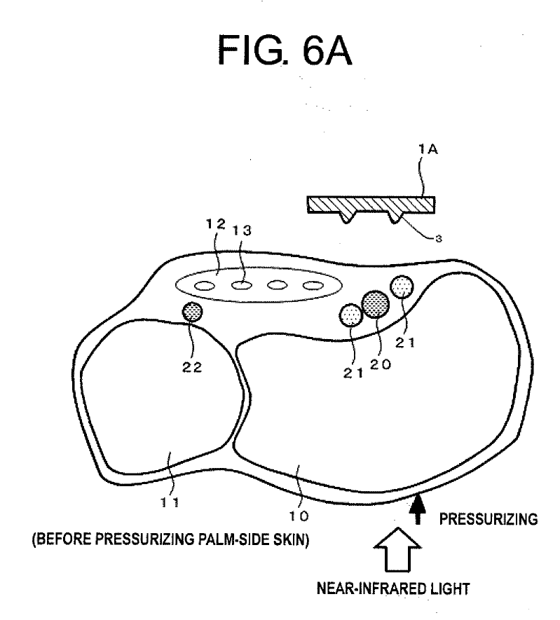

For example, the notch 2 may take the cutting shape of the letter U, V, or I. Furthermore, the notch 2 on a side edge of the plate-shaped material has an opening width L4 and a length L5 in the cutting direction, which can be determined, for example, depending on the diameter of the artery of the portion to be punctured at which the auxiliary tool 1A is used, the puncture direction at the notch 2, the puncture angle to the skin surface, and the thickness L3 of the auxiliary tool 1A. For example, as shown in FIG. 1A, to provide an auxiliary tool preferred for puncture with the running direction of the artery 20 in parallel to the cutting direction of the notch 2, it is preferable that the opening width L4 of the notch 2 be 1 mm to 3 mm, and the length L5 in the cutting direction be 1 mm to 3 mm. On the other hand, as shown in FIG. 4, to provide an auxiliary tool preferred for puncture with the running direction of the artery 20 intersecting the cutting direction of the notch 2, it is preferable that the opening width L4 of the notch 2 be 2 mm to 6 mm, and the length L5 in the cutting direction be 3 mm to 10 mm.

On the bottom surface of the auxiliary tool 1A (i.e., the surface to be in contact with the skin of the portion to be punctured), elongated projections 3 are formed on respective sides of the notch 2 so as to extend in the cutting direction thereof. When the auxiliary tool 1A is pressed against the skin of the portion of the artery 20 to be punctured, the elongated projections 3 act in a manner such that the deep vein 21 running in parallel to the artery 20 can be more effectively pressurized and readily collapsed, and the artery 20 is resistant to displacement in the radial direction thereof.

For this reason, the distance L6 between the tops of the elongated projections 3 on respective sides of the notch 2 is preferably equal to or greater than the diameter of the artery 20 to be punctured. For example, when the artery 20 to be punctured is the radial artery, the distance L6 is preferably 2 mm to 5 mm, and for the brachial artery, the distance L6 is preferably 4 mm to 7 mm. Furthermore, from the viewpoint of effectively pressurizing the deep vein running in parallel to the artery, the height L7 of the elongated projections 3 is preferably 1 mm to 3 mm when the artery to be punctured is the radial artery, and for the brachial artery, the height L7 is preferably 2 mm to 4 mm.

The elongated projections 3 may preferably have a sectional shape of a mountain type with the tops rounded as shown in FIG. 1B.

On the other hand, to puncture the ulnar artery having less deep veins running in parallel thereto, no elongated projections 3 may be provided, and the plane of the auxiliary tool 1A may be formed to be flat.

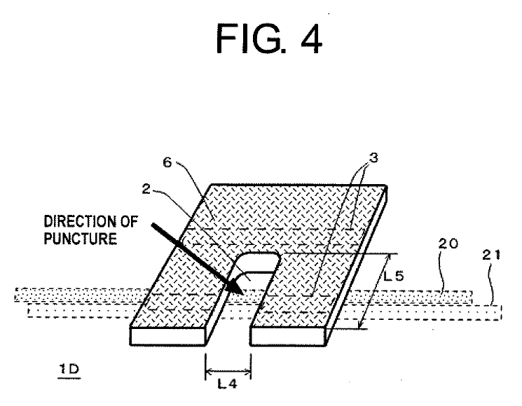

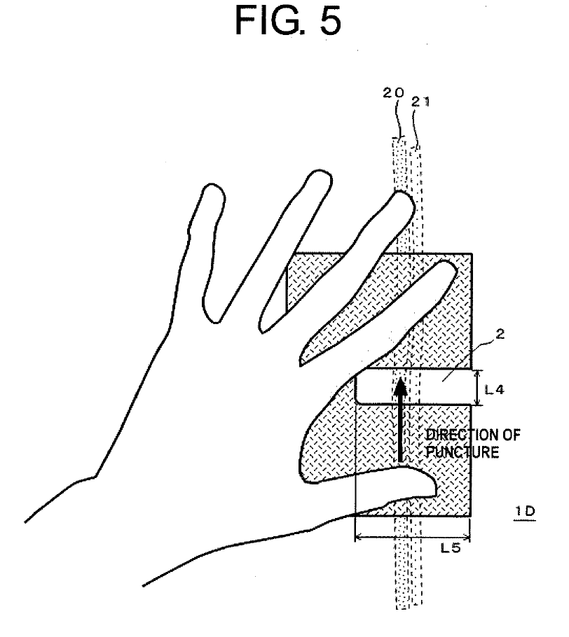

On the upper surface of the auxiliary tool 1A (i.e., the surface with which a finger is brought into contact to depress the auxiliary tool 1A), it is preferable to provide a finger slip prevention mechanism for preventing the slippage of the finger. This makes it possible to readily displace the auxiliary tool 1A with a finger of a puncture operator when, as a method of using the auxiliary tool 1A, the position of the auxiliary tool 1A is displaced with the finger, while the auxiliary tool 1A is being pushed with the finger against the skin of the portion to be punctured, so as to find the position at which the artery is selectively best visualized. As the mechanism for preventing the slippage of the finger, the upper surface of the auxiliary tool 1A may be provided with microscopic asperities formed to prevent the slippage of the finger, or alternatively, provided with an adhesive resin layer. Furthermore, as an auxiliary tool 1B shown in FIG. 2, it is acceptable to form, on both sides of the notch 2, a recess 4 corresponding to the outer shape of a finger depressing the auxiliary tool 1B. Or alternatively, it is acceptable to form, on both sides of the notch 2, a groove 5 into which the belly of a finger is placed as with an auxiliary tool 1C shown in FIG. 3.

Note that in the auxiliary tool of the present invention, the elements such as the presence or absence of the mechanism for preventing the slippage of a finger or the configuration of the mechanism for preventing the slippage of a finger, the presence or absence of the aforementioned notch 2 or the shape and size thereof, the presence or absence of the elongated projections 3 or the shape and size thereof may be combined as appropriate. For example, to facilitate puncture with the running direction of the artery 20 intersecting the cutting direction of the notch 2, an auxiliary tool 1D shown in FIG. 4 is configured such that the opening width L4 of the notch 2 is made wider than that of the auxiliary tool 1A shown in FIG. 1A and FIG. 1B, the bottom surface is formed to be flat, and the entirety of the upper surface 6 is provided with microscopic asperities formed to prevent slippage. Note that the bottom surface of the auxiliary tool 1D shown in FIG. 4 may also be provided with the elongated projections 3 that are similar to those of the auxiliary tools shown in FIG. 1A to FIG. 3. In this case, as illustrated with broken lines in FIG. 4, the elongated projections 3 are preferably provided in parallel to the direction of puncture.

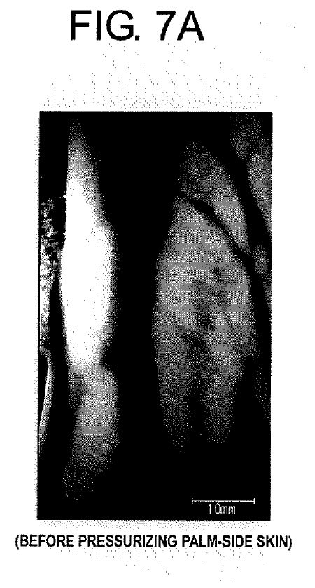

A method for visualizing an artery of the present invention includes: irradiating a portion of an artery to be punctured with near-infrared light and forming a near-infrared image of the portion to be punctured using the reflected light thereof or transmitted light, thereby visualizing the artery. Specifically, the method includes pressurizing the surface of the skin to be punctured with a near-infrared light transparent material so that the deep vein running in parallel to the artery is collapsed, and the subcutaneous vein of the portion to be punctured is collapsed. Since the artery is more difficult to collapse than the vein, the force to pressurize the skin to be punctured at the portion to be punctured may be adjusted as appropriate. This makes it possible to collapse the deep vein running in parallel to the artery and to collapse the subcutaneous vein of the portion to be punctured without collapsing the artery. For example, when the radial artery is to be punctured with the wrist as a portion to be punctured, the backside of the wrist is pressurized by following the method disclosed in Patent Literature 1 or 2 to collapse the capillary network of the skin on the backside of the wrist, and meanwhile, the backside of the wrist is irradiated with near-infrared light so as to form a near-infrared image of the wrist using the transmitted light. At this time, as shown in FIG. 6A and FIG. 6B, the skin surface on the palm side of the wrist serving as the surface of the skin to be punctured is pressurized with the auxiliary tool 1A of the present invention. In this case, the operator to puncture the radial artery aligns the position of the radial artery 20 with the position of the notch 2 so that the running direction of the radial artery 20 is parallel to the cutting direction of the notch 2, and places his or her fingers at the positions indicated by the broken lines 7 on both sides of the notch 2 in FIG. 1A so as to pressurize the skin on the palm side of the wrist with the auxiliary tool 1A. Note that FIG. 6A and FIG. 6B show the radius 10, the ulna 11, the carpal tunnel 12, and the flexor tendon 13.

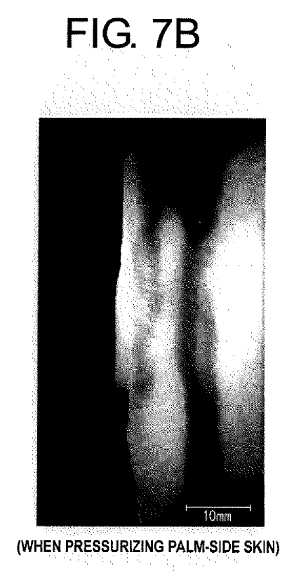

In a near-infrared image before the skin on the palm side is pressurized, even when the capillary network of the skin on the backside of the wrist is collapsed, a blood-vessel image of an increased diameter is observed, in the case of which the blood-vessel image of the radial artery 20 is overlapped with the blood-vessel image of the deep vein 21 running in parallel thereto as shown in FIG. 7A. In contrast to this, as shown in FIG. 6B, the pressurization of the palm side of the wrist allows the deep vein 21 running in parallel to the artery 20 and the subcutaneous vein on the palm side to be collapsed without collapsing the artery 20 as well as the capillary network of the skin on the palm side being pressurized to be collapsed. As shown in FIG. 7B, this causes the blood-vessel images of the collapsed deep vein and subcutaneous vein to disappear in the near-infrared image, and thus the radial artery can be selectively and clearly found as a blood-vessel image that has been reduced in diameter as compared with before the pressurization. In this manner, according to the present invention, the artery 20 can be selectively visualized.

Note that the blood-vessel image of a portion to be punctured in a near-infrared image varies depending on the pressurization of the surface of the skin to be punctured. It is thus preferable that while the surface of the skin to be punctured is being pressurized by depressing the auxiliary tool 1A with a finger of a puncture operator, the position of the auxiliary tool 1A be displaced on the skin surface, the pressurization be released, or the pressurizing force be varied so as to find a near-infrared image in which the blood-vessel image of the artery at the time of pressurization is clearly reduced in diameter as compared with before the pressurization, so that the position of the artery found out in the image is punctured.

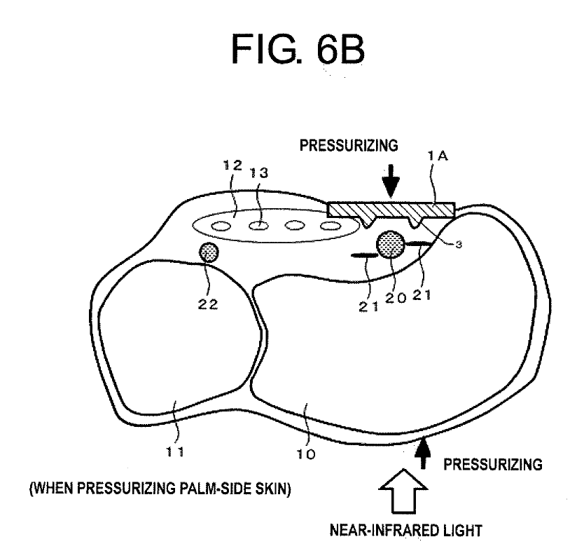

On the other hand, in pressurizing the surface of the skin to be punctured using an auxiliary tool of the present invention, for example, using the auxiliary tool 1D shown in FIG. 4, the skin surface may also be pressurized to puncture the artery 20 while the running direction of the artery 20 intersects the cutting direction of the notch 2 of the auxiliary tool 1D as shown in FIG. 5. In this case, it is preferable that the deep vein 21 running in parallel to the artery 20 be strongly depressed upstream of the vein from the notch 2 so as to puncture the same through the notch 2. This makes it possible to selectively visualize the artery at the time of pressurization when compared with before the pressurization.

The method of the present invention is not limited to the case where the artery to be punctured is the radial artery. Examples of the artery to be punctured other than the radial artery may include the ulnar artery, the brachial artery, the dorsalis pedis artery, and the femoral artery. Here, when the radial artery, the ulnar artery, or the brachial artery is to be punctured, it is assumed that the skin surface to be pressurized with the auxiliary tool of the present invention is the skin surface of the portion to be punctured on the palm side thereof. Furthermore, when the dorsalis pedis artery or the femoral artery is to be punctured, it is assumed that the skin surface to be pressurized is the skin surface of the portion to be punctured on the backside thereof.

### REFERENCE SIGNS LIST

1A, 1B, 1C, 1D auxiliary tool for selectively visualizing artery

2 notch

3 elongated projection

4 recess

5 groove

6 upper surface

10 radius

11 ulna

12 carpal tunnel

13 flexor tendon

20 artery, radial artery

21 deep vein

22 ulnar artery

L1 width

L2 length

L3 thickness

L4 opening width of notch

L5 length in cutting direction of notch

L6 distance between tops of elongated projections on respective sides of notch

L7 height of elongated projection

ΔX amount of distortion

1. An auxiliary tool configured to selectively visualize an artery to be used when forming a near-infrared image of a portion of the artery to be punctured,
the tool being formed from a near-infrared light transparent material and comprising a puncture part through which a puncture needle can pass.

2. The auxiliary tool according to claim 1, comprising a notch as the puncture part.

3. The auxiliary tool according to claim 2, comprising elongated projections extending on a bottom surface of the auxiliary tool in a cutting direction, in which the notch is formed, on respective sides of the notch.

4. The auxiliary tool according to claim 1, comprising a finger slip prevention mechanism, on an upper surface of the auxiliary tool, configured to prevent the finger depressing the upper surface from slipping.

5. A method for visualizing an artery by forming a near-infrared image of a portion of the artery to be punctured, the method comprising:
pressurizing, with a near-infrared light transparent material, a surface of a skin to be punctured so as to collapse a deep vein running in parallel to an artery, and a subcutaneous vein on a puncture side, thereby selectively visualizing the artery.

6. The method for visualizing an artery according to claim 5, wherein the near-infrared image of a portion of the artery to be punctured is formed by transmitted light of near-infrared light with which a surface of a skin opposite to the skin to be punctured is irradiated while the surface of the skin is being pressurized.

7. The auxiliary tool according to claim 2, comprising a finger slip prevention mechanism, on an upper surface of the auxiliary tool, configured to prevent the finger depressing the upper surface from slipping.

8. The auxiliary tool according to claim 3, comprising a finger slip prevention mechanism, on an upper surface of the auxiliary tool, configured to prevent the finger depressing the upper surface from slipping.
